(19) United States
(12) Patent Application Publication
(10) Pub. No.: US 2011/0238070 A1
(43) Pub. Date: Sep. 29, 2011

(54) Orthopedic Downcutting Instrument and Associated Systems and Methods

(75) Inventors: Stephen Santangelo, Wethersfield, CT (US); Ronald G. Litke, Sandy Hook, CT (US); Hanspeter Robert Bayer, Meriden, CT (US)

(73) Assignee: ACCELERATED ORTHOPEDIC TECHNOLOGIES, INC., Fairfield, CT (US)

(21) Appl. No.: 13/036,859

(22) Filed: Feb. 28, 2011

Related U.S. Application Data
(60) Provisional application No. 61/309,057, filed on Mar. 1, 2010.

(51) Int. Cl. A61B 17/16; A61B 17/00

(52) U.S. Cl. 606/80; 606/79

(57) ABSTRACT

Tools and instruments for use in orthopedic applications for forming openings and/or cavities in anatomical structures, e.g., bone and cartilage structures, are provided. The tools/instruments include a shaft having a distal end and defining a longitudinal axis and a shank disposed at the distal end of the shaft. The shank includes a tapered cutting edge and an adjacent fluted region. The shank is effective to collect and deposit materials generated by a cutting action within an opening or cavity formed thereby. A stop may be mounted with respect to the shaft. The tools/instruments deliver a clean and crisp cut to anatomical structures, thereby facilitating interaction with subsequently delivered plugs and/or implants.

### CROSS-REFERENCE TO RELATED APPLICATION

The present application claims the benefit of a co-pending provisional patent application entitled “Orthopedic Downcutting Instrument and Associated Systems and Methods”, which was filed on Mar. 1, 2010, and assigned Ser. No. 61/309,057. The entire contents of the foregoing provisional patent application are incorporated herein by reference.

### FIELD OF THE INVENTION

The present disclosure is directed to tools/instruments for use in orthopedic applications and to systems/methods for forming openings and/or cavities in anatomical structures, e.g., bone and cartilage structures. The present disclosure further relates to advantageous applications of the disclosed tools/instruments, systems and methods in clinical (e.g., orthopedic) applications.

### BACKGROUND OF THE INVENTION

Articular cartilage is a complex structure that, once damaged, has little capacity for permanent repair. One technique that has received attention for addressing cartilage-related issues involves repair with living hyaline cartilage through osteochondral autograft transplant (OAT). This procedure is sometimes referred to as mosaicplasty and generally involves removing injured tissue from a damaged area. One or more cylindrical sockets are drilled into the underlying bone and one or more cylindrical plug grafts—consisting of healthy cartilage from the knee—are implanted in each socket. The foregoing procedures and related technology are described in greater detail in a PCT application filed on behalf of John S. Reach, Jr., entitled “Systems, Devices and Methods for Cartilage and Bone Grafting,” in the U.S. Receiving Office under Certificate of Express Mailing dated May 29, 2009. The foregoing PCT application is incorporated herein in its entirety.

Commercially available instruments for use in OAT procedures include Acufex instruments available from Smith & Nephew, Inc. (Andover, Mass.), the COR System available from Innovasive Technologies (Marlborough, Mass.), and the Arthrex Osteochondral Autograft Transfer System available from Arthrex (Naples, Fla.).

Despite efforts to date, a need remains for instruments, systems and methods for efficient, effective and reliable creation of openings and/or cavities in anatomical structures, e.g., bone and cartilage structures. In addition, a need remains for instruments/systems/methods that facilitate deposit of materials that promote healing in such openings and/or cavities. These and other needs are met by the instruments, systems and associated methods disclosed herein.

### SUMMARY OF THE INVENTION

The present disclosure provides advantageous apparatus, systems and methods for creating openings and/or cavities in anatomical structures, e.g., bone and cartilage structures. The disclosed apparatus deliver a clean and crisp cut to anatomical structures, thereby facilitating interaction with subsequently delivered plugs and/or implants. For example, cartilage implant material may be introduced to openings/cavities formed in a cartilage substrate. In like measure, bone implant material may be introduced to openings/cavities formed in a bone substrate. Thus, the present disclosure is further directed to advantageous clinical (e.g., orthopedic) applications of the disclosed apparatus and systems.

Of note, the disclosed apparatus, systems and methods are effective in depositing materials that promote healing, e.g., bone chips, blood and/or pulp, in such openings and/or cavities during the process of forming such openings/cavities. The deposited materials may be left in place (in whole or in part) to promote healing, e.g., when an implant is positioned in the opening/cavity, or the deposited materials may be harvested (in whole or in part) for use elsewhere in the body. Thus, the design and operation of the disclosed apparatus is generally effective in retaining and/or collecting anatomical materials having potential utility in clinical settings, e.g., without a need for the user to engage in distinct retention and/or collection steps/actions.

Additional features, functions and benefits of the disclosed apparatus, systems and methods will be apparent from the detailed description which follows, particularly when read in conjunction with the accompanying figures.

### BRIEF DESCRIPTION OF THE DRAWINGS

For a more complete understanding of the present invention, reference is made to the following detailed description of exemplary embodiment(s) considered in conjunction with the accompanying drawings, in which:

FIG. 1 is a side perspective view of a first embodiment of the instrument component according to the present invention;

FIG. 2 is a first side view of the instrument component of FIG. 1;

FIG. 3 is a second side view of the instrument component of FIG. 1 opposite the first side view of FIG. 2;

FIG. 4 is a front perspective view showing the instrument component taken along line 4-4 of FIG. 2;

FIG. 5 is a cross-section view of the instrument component taken along line 5-5 of FIG. 3;

FIG. 6 is a front view of the instrument component;

FIG. 7 is a side view of the instrument component taken from the vantage of line 7-7 of FIG. 6;

FIG. 8 is a side perspective view of a second embodiment of an instrument component of the present invention; and

FIG. 9 is a side view of the second embodiment of the instrument component of FIG. 8.

### DETAILED DESCRIPTION OF EXEMPLARY EMBODIMENT(S)

The disclosed tools/instruments, systems and methods are adapted to create openings and/or cavities in anatomical structures, e.g., bone and cartilage structures. The openings/cavities created by the disclosed tools/instruments, systems and methods are generally characterized by clean and crisp edges/walls, thereby facilitating interaction with subsequently delivered plugs and/or implants. The disclosed tools/instruments, systems and methods also generally operate to automatically deposit materials generated during the cutting operation in the opening/cavity created thereby. The deposited materials typically include materials that promote healing, e.g., bone chips, blood and/or pulp, and may be left in place (in whole or in part) and/or harvested (in whole or in part) for use elsewhere in the body.

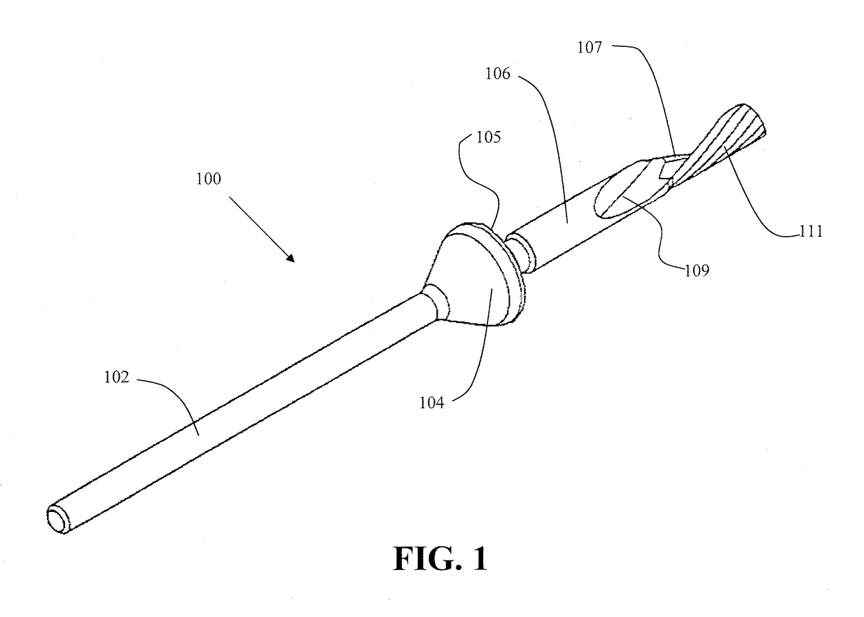

With reference to FIGS. 1-7, an exemplary embodiment of the present disclosure includes an instrument component 100 that includes a mounting shaft portion 102, an intermediate stop 104, and a shank 106. The mounting shaft portion 102 is generally adapted to be removably/detachably mounted with respect to a drive system, as is generally known in the art. Stop 104 defines a substantially conical geometry, which may be formed at an angle of θ², and a substantially planar stop surface 105 that faces distally relative to instrument component 100. Stop 104 generally functions to control/limit distal movement of shank 106 relative to an anatomical structure, e.g., a bone or cartilage structure. Thus, stop 104 may be designed so as to permit/accommodate repositioning along the axial extent of instrument component 100, as will be apparent to persons skilled in the art. Various mechanisms may be employed to adjustably or fixedly secure stop 104 relative to shaft portion 102, e.g., key/slot arrangements, collet arrangements, friction fit arrangements, and the like. Further, the stop 104 may be inherent with the instrument component 100 or may be a modular component separate and apart from instrument component 100. In such an embodiment, different stops 104 may be manufactured based on different needs.

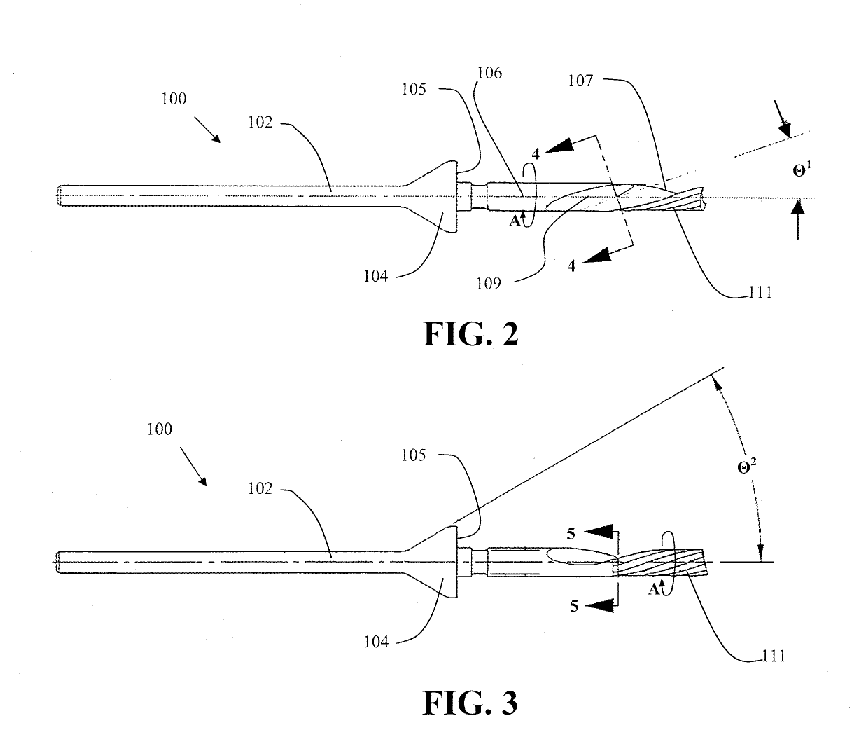

With particular reference to shank 106 of instrument component 100, it is noted with reference to FIGS. 1, 2 and 4 that a tapered cutting edge 107 is defined along an edge surface thereof. The tapered cutting edge 107 is generally helical in shape and continues along an angle θ¹, as shown in FIG. 2. The cutting edge 107 is adjacent to and outward of a fluted region 109 defined in shank 106. The fluted region 109 cooperates with the cutting edge 107 to collect and deposit materials generated by the cutting action of instrument component 100 within the cavity/opening formed thereby. Reliefs 111 may be defined in the outer surface of the shank 106, e.g., reliefs 111 may extend for a distance equal (or substantially equal) to the axial length of cutting edge 107.

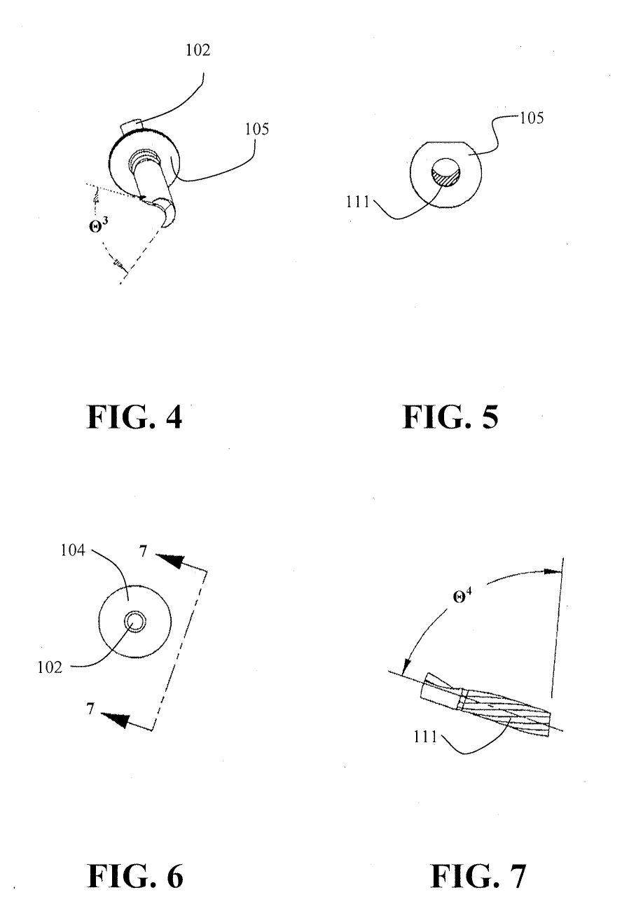

As shown in FIG. 4, the fluted region 109 defines an arcuate opening θ³. In an exemplary embodiment, θ³ may be approximately 70°, although alternative arcuate openings may be employed according to the present disclosure, e.g., from approximately 40° to about 100°. The pitch of the disclosed shank 106, i.e., the axial distance associated with a complete revolution of the shank 106, may also vary from implementation-to-implementation of the present disclosure. Thus, for example, the pitch of an exemplary embodiment of the present disclosure may be approximately 27 mm, although alternative pitches may be employed without departing from the spirit or scope of the present invention.

In exemplary implementations of the present invention, an instrument component 100 may be mounted with respect to a drive mechanism. To the extent the instrument component 100 includes a “stop” structure and to the extent the stop structure is repositionable, e.g., stop 104, the stop may be positioned at a desired cutting depth. The distal end of the shank 106 of the instrument component 100 is positioned adjacent the anatomical structure-of-interest and the drive mechanism is actuated to cause rotational motion of the shank 106. Control of the cutting rpm may be provided, as is known in the art.

Importantly, the cutting edge 107 of instrument component 100 is rotated such that the cutting edge 107 contacts the structure-of-interest along its tapered surface, thereby forming a clean/crisp cut and feeding the cut materials into the fluted region 109 which substantially deposits such materials in the cavity/opening being formed through the noted cutting action. Thus, with reference to FIGS. 2 and 3, the rotational motion is generally in the direction of arrow “A”, i.e., clockwise relative to the longitudinal axis of shaft portion 102 as it extends toward shank 106. Of note, if the structural characteristics of shank 106 are symmetrically reversed, i.e., defined as a mirror image of the structural characteristics shown in the exemplary embodiment of FIGS. 1-7, then the rotational motion of instrument component 100 would be reversed, i.e., counter-clockwise relative to the longitudinal axis of shaft portion 102 as it extends toward shank 106. Thus, the direction of rotational motion of the shaft portion 102 is selected based on the orientation of the cutting edge, such that:
In implementations of the present disclosure wherein the helix of the cutting edge travels in a clockwise orientation relative to the longitudinal axis of the shaft, then the rotational motion of the shaft portion/cutting edge is counter-clockwise; and
In implementations of the present disclosure wherein the helix of the cutting edge travels in a counter-clockwise orientation relative to the longitudinal axis of the shaft, then the rotational motion of the shaft portion/cutting edge is clockwise

Through such structural/geometric configuration and rotational motion, the disclosed tools/instruments, systems and methods are effective in generating clean/crisp cutting edges to the structures-of-interest (e.g., bone and cartilage) and further effective in depositing materials generated by such cutting action in the cavity/opening being formed. Of note, by depositing such materials in the cavity/opening—rather than conveying such materials proximally along the instrument shaft—the potential for chipping and other abrasive effects that can translate into surface irregularities are generally avoided. Specifically, as the instrument component 100 rotates about a longitudinal axis in the direction of arrow “A”, the cutting edge cuts downward relative to the surface being penetrated. Due to the fibrous nature of cartilage, “up-cutting,” i.e. standard cutting, will leave an undesirable edge that is not clean or crisp.

In exemplary clinical applications of the disclosed tools/instruments, the shaft portion 102 is mounted relative to a drive mechanism so as to supply a source of rotational motion thereto. The drive mechanism is associated with a power source, as is well known in the art. The shank 106 of the tool/instrument is then positioned adjacent a desired anatomical location. In exemplary clinical uses, an incision and/or dissection is required to gain access to the desired anatomical location. Alternatively, the disclosed tools/instruments may be used in a minimally invasive manner, e.g., through a cannula, arthroscope or other access device.

Once the shank 106 of the disclosed tool/instrument is brought adjacent to the desired anatomical structure, e.g., bone or cartilage, the drive mechanism is actuated so as to deliver rotational force to the shank 106. As noted above, the direction of rotation is determined by the geometric arrangement of the shank 106 but, in the case of the exemplary embodiment depicted in FIGS. 1-7, the shank 106 is rotated in a clockwise direction to the longitudinal axis of shaft portion 102 as it extends toward shank 106, i.e., in the direction of arrow “A” of FIGS. 2-3.

The advantageous cutting features of the disclosed tool/instrument are effective to create openings and/or cavities in the targeted anatomical structures, e.g., bone and cartilage structures. The openings/cavities are generally characterized by clean and crisp edges/walls, thereby facilitating interaction with subsequently delivered plugs and/or implants. In addition, the cutting action of the disclosed tools/instruments automatically deposit materials generated during the cutting operation in the opening/cavity created thereby. The deposited materials typically include materials that promote healing, e.g., bone chips, blood and/or pulp, and may be left in place (in whole or in part) and/or harvested (in whole or in part) for use elsewhere in the body.

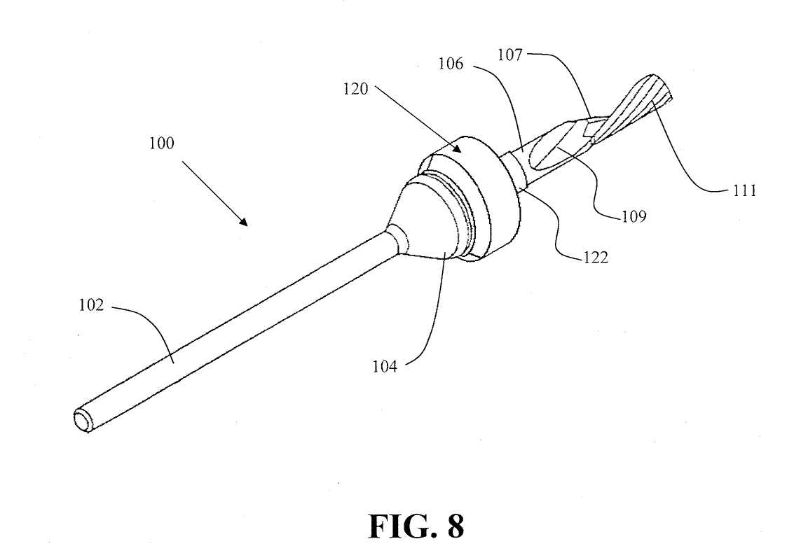

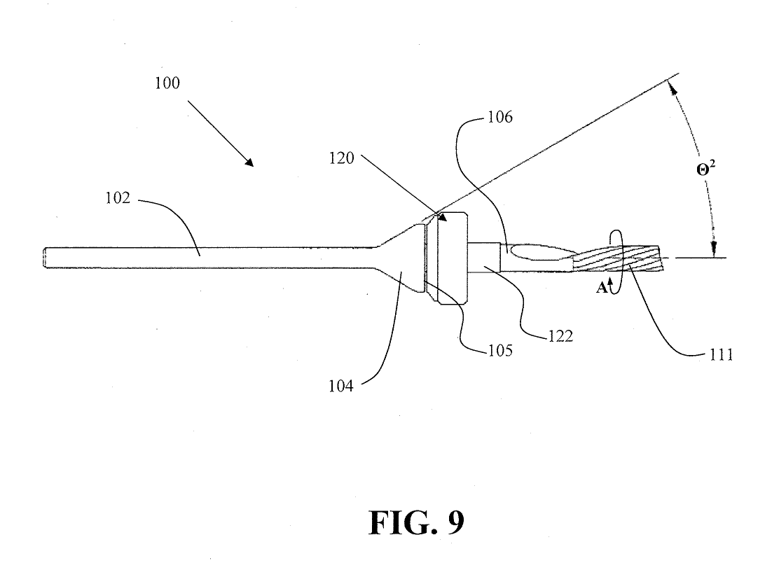

FIGS. 8-9 are respectively a side perspective view and a side view of a second embodiment of the present invention. With the addition of a buffer 120, the disclosed second embodiment includes all of the structural components of the first embodiment and, as such, like numerals are used for like components. The buffer 120 includes a sleeve 122 and is attachable to the shank 106. When attached to the shank 106, the buffer 120 is positioned adjacent to the stop 104 while the sleeve 122 surrounds a portion of the shank 106. The buffer 120 may be adjustable or fixed in place relative to the shank 106 dependent upon desired construction and use. Various mechanisms may be employed to secure buffer 120 relative to the shank 106, e.g., key/slot arrangements, collet arrangements, friction fit arrangements, and the like.

The buffer 120 may be compressible and generally functions to provide a cushion upon full insertion of the shank 106 into an anatomical structure during clinical use of the disclosed apparatus/systems. Specifically, the stop 104 can be positioned to a desired cutting depth and the buffer 120 positioned so that the planar surface 105 of the stop 104 does not forcefully engage the anatomical structure, but rather the buffer 120 engages the anatomical structure and provides a cushioned stop. Even further, the buffer 120 may function as a stop whereby a user can set the buffer 120 to a desired cutting depth and then choose to provide a deeper cut when at the set depth by compressing the buffer 120.

Although the tools/instruments, systems and methods of the present disclosure have been described with reference to exemplary embodiments, the present disclosure is not limited by or to such exemplary embodiments. Rather, the tools/instruments, systems and methods of the present disclosure are susceptible to many variations, modifications and/or implementations without departing from the spirit or scope of the present disclosure.

What is claimed is:

1. An orthopedic apparatus for cutting tissue, the apparatus comprising:
a shaft having a distal end and defining a longitudinal axis; and
a shank disposed at the distal end of said shaft, the shank including a tapered cutting edge and an adjacent fluted region;
wherein said shank is effective to collect and deposit materials generated by a cutting action within an opening or cavity formed thereby.

2. The orthopedic apparatus of claim 1, wherein said shaft is mountable with respect to a drive mechanism.

3. The orthopedic apparatus of claim 1, further comprising a stop mounted to said shaft.

4. The orthopedic apparatus of claim 3, wherein said stop is fixedly mounted to said shaft.

5. The orthopedic apparatus of claim 3, wherein said stop is modular and separate from said shaft.

6. The orthopedic apparatus of claim 3, wherein said stop is adjustably mounted with respect to said shaft.

7. The orthopedic apparatus of claim 6, wherein said stop can be rotationally or axially adjusted with respect to said shaft.

8. The orthopedic apparatus of claim 1, wherein said shaft rotates in a clockwise direction relative to the longitudinal axis of said shaft.

9. The orthopedic apparatus of claim 1, wherein at least one relief is defined on an outer surface of said shank.

10. The orthopedic apparatus of claim 1, wherein said fluted region defines an arcuate opening when viewed from the distal end of said shaft.

11. The orthopedic apparatus of claim 10, wherein the angle of said arcuate opening is in the range of about 40° to about 100°.

12. The orthopedic apparatus of claim 1, wherein said shank defines a pitch of 27 mm.

13. The orthopedic apparatus of claim 1, further comprising a buffer mounted with respect to said shaft.

14. The orthopedic apparatus of claim 13, further comprising a stop mounted with respect to said shaft, and wherein said buffer is positioned adjacent to said stop.

15. The orthopedic apparatus of claim 13, wherein said buffer is compressible.

16. A method for forming an opening or cavity in an orthopedic application, the method comprising:
providing an apparatus, the apparatus including:
a shaft having a distal end and defining a longitudinal axis; and
a shank disposed at the distal end of said shaft, the shank including a tapered cutting edge and an adjacent fluted region;
placing said apparatus adjacent to a substrate; and
rotating said apparatus so that said tapered cutting edge engages said substrate to form an opening or cavity in said substrate;
wherein said fluted region collects and deposits materials generated by said cutting action within the opening or cavity formed in said substrate.

17. The method of claim 16, wherein said substrate is selected from the group consisting of bone, cartilage or a combination of bone and cartilage.

18. The method of claim 16, wherein said deposited materials are selected from the group consisting of bone chips, blood, pulp and combinations thereof.

19. The method of claim 16, further comprising harvesting said deposited materials from the opening or cavity, in whole or in part, for clinical use.

20. The method of claim 16, further comprising inserting a plug or implant into said opening or cavity.
